(19) United States
(12) Patent Application Publication
(10) Pub. No.: US 2020/0164166 A1
(43) Pub. Date: May 28, 2020

(54) VENTILATOR

(71) Applicant: LIFELINE TECHNOLOGIES LIMITED, Colchester (GB)

(72) Inventor: Alastair Rupert Joseph DARWOOD, London (GB)

(21) Appl. No.: 16/632,112

(22) PCT Filed: Jul. 7, 2018
(86) PCT No.: PCT/GB2018/052029

§ 371 Date: Jan. 17, 2020

(30) Foreign Application Priority Data
Jul. 17, 2017 (GB) 1711495.0
Dec. 14, 2017 (GB) 1720920.6

(51) Int. Cl. A61M 16/00; A61M 16/20

(52) U.S. Cl. CPC A61M 16/022; A61M 16/205; A61M 2205/15; A61M 2205/70; A61M 2016/0027; A61M 2205/18; A61M 2230/46

(57) ABSTRACT

Apparatus for a ventilator, comprises a control means (108) configured to: receive state information indicative of pressure being above or below at least one predetermined threshold pressure at at least one pressure switch (208), and control an air movement device to repeatedly cause inflation of lungs of the patient and allow deflation, dependent at least on the received state information. In use, the at least one pressure switch is located at a pressure representative of air pressure in lungs (202) of a patient. The state information does not comprise an absolute numerical pressure value at the at least one pressure switch.

### FIELD OF THE INVENTION

The invention relates to apparatus for a medical ventilator. The invention also relates to a method of operation of a medical ventilator and a computer program product therefor.

### BACKGROUND

Both electrical and pneumatic powered ventilators, and combinations thereof, are known in the art. Ventilator designs also include entirely mechanical and ‘human powered’ systems. Smaller, portable devices lend themselves to remote or trauma situations such as first response care, whilst more complex feature rich devices are used in settings such as intensive care units to fully manage a patient's respiration in the long term.

Current devices provide safe, reliable ventilation if well maintained and can provide in depth information relating to patient ventilation such as real time gas volumes and pressures including graphing of flow/volume curves over time. These features necessitate the use of digital pressure and flow sensing technologies including associated software, which introduces several well recognised drawbacks. Medical grade digital pressure and flow sensors are costly to manufacturers due to accuracy and reliability requirements. In addition, delicate electronics and sensors decrease robustness of digital ventilators shortening their maintenance free lifespan, and making them less rugged. Complex software may additionally be needed to integrate all sensor outputs with ventilator function. This may increase both up front and operating costs and means that devices are relatively fragile and expensive to maintain, and may require calibration procedures to ensure continued accuracy.

Pneumatic ventilator systems usually consist of a device with a pressurised oxygen or air source input connected to a regulator system that generates periodic air flow to a patient. These systems can be entirely mechanical using the gas pressure to ‘power’ the device or ‘electromechanical’ using digital sensors to monitor respiratory parameters but pneumatic apparatus to provide air flow. Whilst pneumatic systems are often less costly than fully digital electronic/electrical ventilators, they have their own set of drawbacks. A constant source of compressed gas is required for device operation in addition to providing oxygen for the patient resulting in an inefficient use of gas. In many situations, compressed oxygen or air is not readily available, may be inconvenient to transport, or in short supply. Whilst more economical than conventional electronic/electrical ventilators, the regulation of compressed gas to periodic, controlled gas flow at respiratory pressures requires complex valves, seals and other sensitive pneumatic components requiring regular upkeep and maintenance whilst rendering the ventilators vulnerable to impact and shock damage.

As a basic level of function, both pneumatic and electronic/electrical ventilator technologies can provide IPPV (intermittent positive-pressure ventilation) allowing an operator to set up the required parameters.

More complex, particularly electronic ventilators, may also provide other respiratory modalities and feedback information such as lung compliance curves and tidal volume controlled ventilation.

In general, it is preferable for any powered ventilation system to possess key features for safe, reliable use:

1. Tidal volume measurement or an indicator for a change in tidal volume or lung compliance.

2. Audible/visual alarms for any errors in the breathing circuit e.g. an air leak, or an over/under pressure situation.

‘Tidal volume’ is defined as the total volume of air moved into and out of a patient's lungs during each successive breath. This is vital information during IPPV as certain lung pathology can cause a decrease in tidal volume despite constant pressures being generated at a patient's airway. This necessitates an increase in the IPPV maximum pressure to ensure an acceptable tidal volume. Ventilation pressures must be tightly controlled, as in an apnoeic patient, excessive pressures can cause lung over-inflation injury, ‘barotrauma’, with significant associated morbidity and mortality.

Alternatives to electronic/electrical and pneumatic ventilators include the manually operated bag-valve-mask (BVM) with multiple derivatives in the art. This consists of a manually compressible self-expanding air bladder that connects to a patient airway interface such as a facemask, laryngeal airway or endotracheal tube. Once connected to a patient airway interface, an operator may squeeze the bag to provide positive pressure to the patient's lungs causing expansion. Whilst low cost, portable and universal, BVM ventilation is often criticised for several reasons: patient overventilation and barotrauma are well recognised problems whilst the approach mandates that a trained attender constantly manually ventilates the patient taking them away from other jobs where their skills may be better used. This is especially problematic if a single clinician is in attendance and patient ventilation is required.

### SUMMARY OF THE INVENTION

In accordance with a first aspect of the present invention, there is provided apparatus for a ventilator, comprising a control means configured to: receive state information indicative of pressure being above or below at least one predetermined threshold pressure at at least one pressure switch, wherein, in use, the at least one pressure switch is located at a pressure representative of air pressure in lungs of a patient, and wherein the state information does not comprise an absolute numerical pressure value indicating the pressure; control an air movement device to repeatedly cause inflation of lungs of the patient and allow deflation, dependent at least on the received state information.

Since a very simple pressure switch may be provided, the cost may be very low. This enables parts of the ventilator that become contaminated in use by the patient's breath to be single use and thrown away after use. Also, such a switch is much less likely to fail than a complex switch configured to monitor air pressure values. Reliability of such a pressure switch means that a ventilator that operates using it may not require checking for a long period and facilitates availability in remote locations. Further, the apparatus may be included in a complex ventilator as a back-up.

The apparatus may comprise a first part and a second part, wherein the first part includes the control means and the air movement device, and the second part includes the at least one pressure switch. In this case the second part is preferably detachably attachable to the first part. Advantageously, any bacterial contamination from the patient may be limited to location on the second part. Alternatively, the first and second parts may be integrated and thus permanently attached.

The apparatus may further comprise means for conveying the state information from the at least one pressure switch to the control means. The state information conveying means may comprise a first information conveying portion in the first part and a second information conveying portion in the second part. In this case the first and second information conveying portions are preferably detachably attachable, which facilitates disposability of the second part.

The apparatus may further comprise conduit means comprising: a first conduit part in the first part of the ventilator coupled to the air movement device; and a second conduit part in the second part of the ventilator, wherein the first and second conduit parts are connectable such that air can be caused to flow from the first conduit part to the second conduit part, and wherein the first and second conduit parts are detachably attachable. The second part may further comprise a patient interface means coupled to the second conduit part and configured for attaching to the patient such that air can be caused to flow into the lungs of the patient. The at least one pressure switch may be located in the patient interface means.

The second part may comprise: a valve means in the second conduit part; and an air outlet coupled to the valve means, wherein the valve means is configured to permit flow of air from a first side of the valve means nearer to the air movement device to a second side of the valve means nearer to the patient when air pressure at the first side is greater than at the second side, and to cause air to flow from the second side through the air outlet when air pressure at the second side is greater than air pressure at the first side. Such valve means may alternatively be included in the patient interface means with the air outlet coupled to the value means.

The apparatus may further comprise a first user control coupled to the at least one pressure switch for setting the at least one threshold pressure. Alternatively, the at least one threshold pressure may be pre-configured and the first user control may be absent.

The apparatus may further comprise a second user control coupled to the control means, configured to enable input of information indicative of at least one of: breathing rate, inspiration period, expiration period, and an inspiration-to-expiration ratio, wherein the control means is configured to store the input information, and to control the air movement device to repeatedly cause inflation of lungs of the patient and allow deflation, dependent also on the stored information.

The at least one threshold pressure may comprise a first threshold pressure, wherein the control means is configured to repeatedly: control the air movement device to cause inflation of the lungs and to start the inspiration period; in response to the state information indicating that the pressure is above the first threshold pressure, determine that the inspiration period is ended, control the air movement device to enable expiration, and start the expiration period; determine that the expiration period has ended based at least one stored information, and in response thereto to repeat.

The at least one threshold pressure may comprise at least two threshold pressures, wherein the state information indicates that pressure is above or below each of the at least two threshold pressures. The at least one pressure switch may be a single switch configured to provide state information indicative of pressure being above or below the at least two thresholds or an arrangement of a plurality of pressure switches each configured to provide state information indicative of the pressure being above or below a respective one of the threshold pressures. The further threshold may trigger, for example, in the event of a malfunction.

The at least two thresholds may comprise a further threshold above the first threshold pressure, wherein, when the control means receives state information indicating that the pressure is above the further threshold, the control means is configured to cause at least one action to be taken. For example, the control means may cause an alert to be communicated to an operator of the ventilator and/or to cause depressurisation, thereby to enable deflation. The further threshold may trigger, for example, in the event of a malfunction not indicating that the first threshold has been passed.

Additionally or alternatively, the at least two threshold pressures may comprise a yet further threshold value below the threshold pressure, the yet further threshold pressure being below the first threshold pressure. The yet further threshold pressure may be indicative of the patient attempting inhalation. In this case, further to receiving state information indicating that the pressure is below the threshold pressure, the control means is configured to cause at least one action to be taken. For example, the control means may cause an alert to be communicated to an operator of the ventilator and/or for the maximum power provided to the air control device to be reduced.

The control means may be further configured to determine duration of the inspiration period.

In a calibration phase, the control means may be further configured to determine a maximum power output for use with the air movement device for the patient, in which case the control means is configured to: when the pressure is below the first threshold pressure, control the air movement device to cause pressure between the air movement device and the patient to rise by increasing the power supplied to the air movement device;

in response to receiving state information indicating that the pressure exceeds the threshold pressure, set the maximum power output of the air movement device as the maximum power for use with the air movement device with the patient. The maximum power output may be configured as a maximum torque or a maximum motor speed, for example.

Information indicative of predetermined breathing rate is preferably stored at the control means. In this case, the inspiration and/or expiration time periods may be dependent on the predetermined breathing rate.

According to a second aspect of the present invention, there is provided a computer program product comprising computer program code stored on a memory means, which, when run on a processing means is configured to cause the processing means to perform the steps of: periodically processing received state information indicative of whether pressure is above or below at least one predetermined threshold pressure at at least one pressure switch, wherein, in use, the at least one pressure switch is located at a pressure representative of air pressure in lungs of a patient, and wherein the state information does not comprise an absolute numerical pressure value indicating the pressure at the at least one pressure switch; controlling an air movement device to repeatedly cause inflation of lungs of a patient and allow deflation dependent at least on the received state information.

According to a third aspect of the present invention, there is provided a method of operation of a ventilator, comprising: periodically processing, at a control means, information from a pressure switch indicative of whether pressure is above or below a predetermined threshold pressure, wherein the pressure sensor is located in air at a pressure representative of air pressure in lungs of a patient, and wherein the information does not comprise an absolute numerical pressure at the pressure sensor; controlling an air movement device to repeatedly cause inflation of lungs of a patient and allow deflation dependent at least on the received information.

### BRIEF DESCRIPTION OF THE FIGURES

For better understanding of the present invention, embodiments will now be described, by way of example only, with reference to the following Figures, in which:

FIG. 1 is a diagram indicating components of a first part of a ventilator in accordance with embodiments of the invention, the first part being in the form of a pressure generating unit;

FIG. 2 is a diagram indicating a patient's lungs, and components of a second part of the ventilator, in accordance with the embodiments;

FIG. 3 indicates diagrammatically the second part connected to the pressure generating unit and coupled to the patient to enable air to be pushed into the patient's lungs;

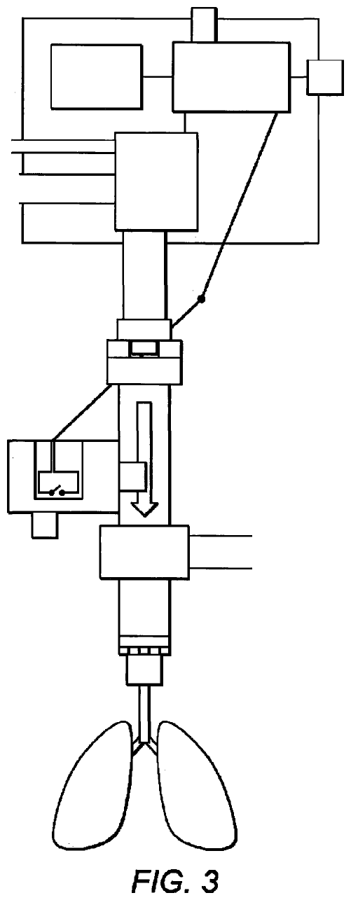

FIG. 4 is a flow chart indicating steps involved in a process of calibrating the ventilator for use with a particular patient;

FIG. 5 is a flow chart indicating step involved in a process of operation of the ventilator; and

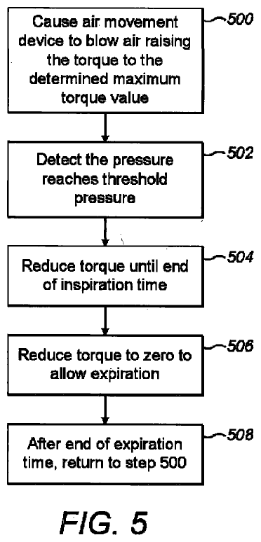

FIG. 6 is a graph indicating pressure in at pressure switch during ventilation, the pressure at the pressure switch being representative of pressure in the lungs.

### DETAILED DESCRIPTION OF EMBODIMENTS

Embodiments of the invention relate to a medical ventilator including an air movement device, a pressure sensitive switch and a controller. The switch is configured with a threshold pressure and to respond to pressure being above or below the threshold pressure by sending a signal indicative of such to a controller. The controller is configured to control the air movement device to repeatedly cause inflation and allow deflation dependent at least on information in the signal. The signal does not have to indicate an absolute value for the pressure; the signal only has to indicate if the signal is above or below the threshold pressure.

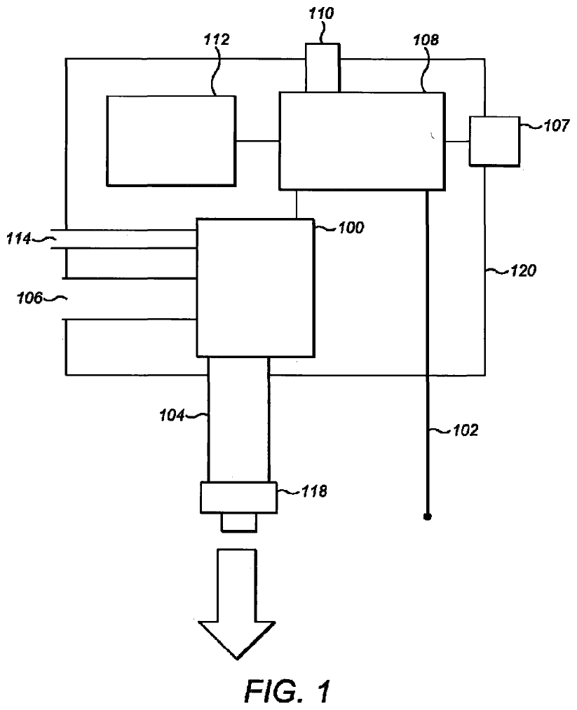

Referring to FIG. 1, a first part of ventilator comprising the pressure generating unit comprises an air movement device 100, a first electric interface 102, a first conduit 104, a first user control 107, the controller 108, a user indication unit 110, a power source 112, an air intake 114, an air outlet 116, a first mechanical attachment part 118, and a housing 120.

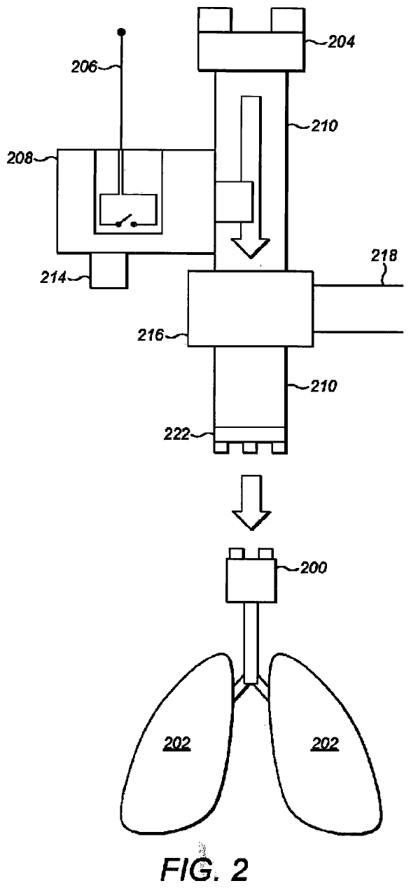

Referring to FIG. 2, a second part comprises a coupling component for coupling the pressure generating unit and a patient airway interface 200, such that air can be pushed from the pressure generating unit into a patient's lungs 202 and also such that exhalation is enabled. The coupling component comprises a second mechanical attachment part 204, a second electrical interface 206, the pressure sensitive switch 208, a housing (not shown), a second conduit 210, and a second user control in the form a dial 214.

Various ways are known in the art to attach a ventilator to a patient. For example, the ventilator can attach over the patient's mouth. In this case, the patient airway interface 200 is a facemask that fits over the patient's mouth, a laryngeal mask airway or endotracheal tube. Embodiments of the invention are not limited to any particular place at or means by which the ventilator attaches to the patient to provide air into the patient's lungs, and, accordingly, the term “patient air interface” is to be construed broadly.

The air movement device 100 is sealingly coupled to the first conduit 104 and controllable by the controller 108 to cause air flow into the first conduit 104. The air movement device 100 is in the form of a centrifugal fan powered by an electric motor, but embodiments of the invention are not limited to any particular way in which the air movement device 100 causes movement of air into the first conduit 104. For example, the air movement device may comprise an impeller or radial fan in place of the centrifugal fan.

The air movement device 100 is connected to the air intake 106 at which atmospheric air is taken into the pressure generating unit for the air movement device 100. The air intake 106 preferably includes an air filter (not shown).

A second intake 114 including a low pressure gas input port, connected to a source of oxygen or other gas. The air movement device 100 is also connected to the second intake 114 to draw oxygen or the other gas into the first conduit 104. In this case, the air and the oxygen or other gas are drawn in and mixed. For example, where the gas is oxygen, the oxygen supplements the atmospheric air to increase the percentage of oxygen gas delivered to the patient with each breath. In a variant embodiment, the second intake 114 may be absent and the ventilator may supply only atmospheric air to the patient.

In addition and although not shown in the figures, the pressure generating unit may also have powered and/or non-powered accessory ports to allow connection of adjuvant hardware, such as an air humidification and heating system or an anaesthetic gas vaporiser.

The controller 108 includes a microcontroller and an electronic speed controller. The electronic speed controller is configured to control torque of the electric motor and thus to control air flow rate. The controller 108 may be configured to control the electronic speed controller to control the air movement device 100 to cause the motor to operate with torque at any value between 0 and 100% of a predetermined maximum flow rate. Optionally, the electronic speed controller is capable of electronically braking the air movement device 100, thus reducing the motor speed in the first conduit 104 to zero very rapidly, resulting in pressure dropping to a minimal value compared to atmospheric pressure. In variant embodiments, functionality of the electronic speed controller is integrated into the microcontroller and thus the electronic speed controller is not needed. In variant embodiments, the speed of the motor may be controlled instead of the torque of the motor. Embodiments of the invention are not limited to any particular parameter of an air movement device that may be controlled to control flow rate.

The microcontroller is configured to control the electronic speed controller and also provides the rest of the functionality ascribed to the controller 108 herein. The microcontroller comprises a central processing unit, a memory unit (readable and writable), an input/output port, and a clock, all operatively connected. A computer program comprising computer program code is stored on the memory unit. Alternatively, dedicated hardware with embedded functionality may be used. Execution of the computer program code by the central processing unit results in the functionality ascribed to the microcontroller. Algorithms implemented in the computer program code are described in greater detail below.

The controller 108 is thus operatively coupled to the air movement device 100 to control flow of air caused by the air movement device 100. The controller 108 is also coupled to the power source 112, for supply of power to the controller 108 and so that the controller 108 can control supply of power to the air movement device 100. The controller 108 is also operatively coupled to the first user interface 107, to the user indication unit 110 and to the first electric interface 102.

The power source 112 may be a battery. Alternatively, the power source 112 may comprise a plug for connecting to a mains power supply.

The first user control 107 is configured to enable the operator to start and stop operation of the ventilator. The first user control 107 is also configured to enable the operator to set one or more variables relating to ventilation, such as breath rate and/or inspiration time. The controller 108 may have default values stored for these variables.

The user indication unit 110 is controllable by the controller 108 to indicate information to the operator. The user indication unit 110 may comprise one or more lights, such as LEDS, and/or include a speaker configured to make a sound audible to the operator of the ventilator. The user indication unit 110 may include a display for indicating to the operator information indicative of the output power of the air movement device, or the values for one or more other variables input by the user using the first user control 107.

The first electric interface 102 comprises an electrical cable connected to the controller 108 at one end and a first electrical attachment at the other end. In the coupling component, the switch 208 is connected to the second electrical interface 206. The second electrical interface 206 comprises an electrical cable connected to the switch 208 at one end thereof, and a second electrical attachment at the other end. The first electrical attachment of the first electrical interface 102 and the second electrical attachment are configured to electrically connect, to carry signals from the switch 208 to the controller 108. The first and second electrical attachments may be, respectively, in the form of a plug and socket each having an electric contact.

In a variant embodiment, instead of the switch 208 and the controller 108 being connected by the first and second electrical interfaces, other means for carrying a signal from the switch 208 to the controller 108 may be provided. For example, the coupling component may include an RF transmitter and an additional microcontroller coupled to the switch 208, and there may be an RF receiver coupled to the controller 108. Thus, the microcontroller can cause the RF transmitter to send information provided by the switch 208, which is received by the RF receiver and passed to the controller 108. Embodiments of the invention are not limited to any particular means by which information obtained from the switch 208 to the controller 108.

The first conduit 104 is sealingly coupled to the air movement device 100 such that operation of the air movement device 100 causes movement of air into the first conduit 104. The first mechanical attachment part is located at an end of the first conduit 104 remote from the air movement device 100. The second mechanical attachment part 204 is attached to an end of the second conduit 210. The first and second mechanical attachment parts are mutually connectable so that air from the first conduit 104 flows into the second conduit 210. The other end of the second conduit 210 provides an outlet that is sealingly and releasably attached to the patient, such that air can be forced into the patient's lungs 202 via the patient airway interface 200. The first and second mechanical attachment parts may be configured to connect in any conventional way, for example with a clip arrangement or other attachment mechanism.

The first and second conduits 104, 210 thus serve to both provide a channel for air pushed by the air movement device 100 and include means for attaching the pressure generating unit and the coupling component. In a variant embodiment, a mechanical connection between the first and second conduits 104, 210 may serve to attach the conduits to provide such a channel without separate mechanical connections being provided for the pressure generating unit and the coupling component. However, the pressure generating unit and the coupling component may be additionally mechanically attached so that they are securely fastened together.

The second conduit 210 has a valve 220 located in it that allows air from the air movement device 100 to the patient's lungs 202, but prevents movement in the second conduit 210 in the opposite direction. Thus, the valve prevents exhaled gas from accessing the pressure generating unit, such that the pressure generating unit does not become contaminated by the exhaled gas. The exhaust port 218 is connected to the valve 220 for venting exhaled gas to the atmosphere or for connection to other apparatus for collecting exhaled gas. When the air pressure in the second conduit 210 is greater in a first side of the valve 220 nearer the patient than on a second side nearer the second mechanical attachment part 204, the valve 220 is configured to allow the exhaled gas from the first side to escape through the exhaust port 218, while preventing the exhaled gas from passing to the second side. Thus, exhalated air can escape from the ventilator. When the pressure is greater on the first side, the value 220 is configured to enable air to pass through the value 220 while blocking the exhaust port 218 to prevent escape of the air.

In other words, the valve 220 is such that when pressure in the first conduit 104 exceeds intrathoracic pressure, the exhaust port 218 is blocked forcing all air flow to enter the lungs via the patient airway interface 200. Once intrathoracic pressure exceeds coupling component pressure, for example when the air movement device 100 is turned ‘off’ during exhalation the exhaust port 218 is unblocked and airflow is directed to the atmosphere via the exhaust port 218.

The switch 208 is either in or in fluid communication with the interior of the second conduit 210 between the valve 220 and the air movement device 100. Thus, the switch 208 can respond to pressure in the second conduit 210, which is considered representative of pressure in the patient's lungs 202. Alternatively, the switch 208 is either in or in fluid communication with an interior of the second conduit between the value 220 and the lungs 200. In embodiments, the switch 208 is located in the patient airway interface 202.

The switch 208 is configured to respond to pressure rising beyond a threshold pressure by sending a signal to the controller 108 indicative of such. The switch 208 is coupled to the dial 214 to enable the threshold pressure to be configured by the operator of the ventilator. The dial 214 can be locked at the selected pressure threshold. Information may be provided on a label adjacent the dial 214 to aid the operator in selecting an appropriate threshold pressure. The appropriate threshold pressure may be dependent on any of the size, age and health of the patient, for example.

The threshold pressure is the target maximum pressure to be achieved during each breath the ventilator provides. Thus, the operator can alter the pressure threshold at which its state is changed (the switch ‘trips’) or changes from one signal state to another. It is stressed that in embodiments the numerical value of pressure threshold of the pressure sensitive switch 208 setting may remain unknown to the controller 108, as the switch 208 provides to the controller 108 information about the switch status, indicative of whether the pressure is above or below the threshold pressure. An actual pressure value is not provided to the controller 108.

The ventilator is operable to move air into lungs by actuating the air movement device 100 to cause a positive air pressure at the patient airway interface 200 with air flow traveling from the air movement device 100, through the coupling component and thence to the patient airway interface 200 and the patient's lungs 202. When this positive air pressure is greater than the intrathoracic pressure, air will move into the lungs resulting in lung expansion and forced inspiration. As the lungs fill, they are increasingly distended thus providing increasing resistance to filling as the force of elastic recoil from lung tissue and chest wall increases proportional to lung distention. When the threshold pressure that the switch 208 is configured to detect is reached, the lungs are distended in accordance with that pressure, and the air movement device 100 is switched off (or reduced to a set minimum level) such that the pressure generated by the pressure generating unit is reduced. As soon as the pressure in the second conduit 210 is less than the intrathoracic pressure, the elastic recoil of lung tissues and chest causes air to flow out of the lungs to the lower pressure atmosphere via the exhaust port 218 until the lungs return to their ‘relaxed’ state and airflow ceases as the intrathoracic pressure is equal to atmospheric pressure. This exhalation process is entirely passive.

The controller 108 is configured to receive a binary signal from the switch 208 representing whether or not the detected pressure is above the pressure threshold or below the pressure threshold. As only two options are available (above or below) there are only two possible signal states. This signal may be an electrical ON/OFF, for example with ‘OFF’ representing ‘below threshold pressure and ‘ON’ representing above threshold pressure.

The first user control 107 enables the operator to input ventilation variables such as desired ventilation rate and inspiratory or expiratory time or ratio. The first user control 107 is not limited to any particular form; the first user control 107 may comprise one or more switches, and/or one or more dials and/or a graphical user interface touchscreen, to directly select a desired input variable. The first user control 107 may be configured to enable selection of one of a predetermined number of values for the or each variable. In a variant embodiment, the first user control 107 may be absent. In this case, the first user control may be replaced with means for connecting to a paired device and the variables may be selected using the paired device. For example, the paired device may be a connected smartphone, tablet or computer, and the connection may use Bluetooth®.

The pressure generating unit is preferably reusable. The coupling component is preferably disposable. The coupling component is intended to be inexpensive to manufacture. Embodiments of the invention are not limited to such; alternatively the coupling component may be reused. Also, it is not essential to the invention that the first and second parts are detachable from one another, which facilitates disposability of the coupling component.

In a variant embodiment, one or more further pressure switches may be provided. Each of the further pressure switches may have a respective user control, such as a lockable dial, to enable the operator to set a threshold pressure. Each of the further pressure switches is configured to provide information indicative of whether the air pressure is above or below the set threshold and not to provide an actual absolute numerical value. In variant embodiments, the threshold for one, any or all further switches may be preconfigured.

As mentioned above, the switch 208 is positioned to detect air pressure in the second conduit 210 on the first side of the valve 220 or, alternatively, the switch 208 may be on the second side of the valve 220 positioned to detect air pressure just prior to the patient air interface 200. As mentioned above, the switch 208 may be ‘binary’ in nature, such that it can determine two values; one value at all pressures lower than the threshold pressure specified on the dial and the other at all pressures greater than the specified value. Determined values may include but are not limited to electrical resistance, ‘OFF’ or ‘ON’ switch output, radio frequency signal or any other ‘status indicating or signalling mechanism. The threshold pressure value may be manually set by an operator with the aid of the dial 214, as mentioned above, or other alternative adjustment mechanism or device. It is understood by those skilled in the art that a suitable pressure sensitive switch may be constructed in a multitude of configurations such that a parameter reliably varies in response to a change in pressure at a user variable threshold value.

In an example, the pressure sensitive switch 208 may consist of a conductive ‘ball’ making a seal over a conductive orifice in the side of the coupling component at the point where pressure is measured. The ball and the orifice are electrically connected to the controller 108 in the pressure generating unit. The ball is pressed onto the orifice with a force provided by a spring. The force provided by the spring is adjustable by rotating a dial coupled to the spring with, for example, a threaded screw. At all pressures lower than a specified pressure, the ball remains in contact with the orifice. At pressures higher than the specified value, the force generated is able to overcome the force of the spring, thus lifting the ball from the orifice and changing the electrical parameters of the system such as increasing the electrical resistance. Alternatives may include a force sensitive resistor in the chamber above the seal or a diaphragm making electrical contact at a user specified pressure.

In alternative embodiments the pressure sensitive switch 208 may be configured with two or more pressure thresholds. One or more of the thresholds may be independently adjustable by the operator, or one or more may be fixed. Thus, three different signal states are possible and may be provided to the controller 108. This may alternatively be implemented by two or more switches each with binary outputs. No actual numerical pressure values are determined.

In certain embodiments, it may be desirable to provide a coupling component with more than one pressure sensitive switch 208 purely for redundancy. Considering the importance of accurate patient airway pressure measurement, multiple pressure sensitive switches provide redundancy should any given switch fail to function correctly. Multiple switches may be provided in differing configurations. For example, two or more identical switches may be used such that at all times the expected output of both switches 208 should be identical. If varied, a switch malfunction can be inferred by the controller 108 and appropriate action taken, such as a return to a calibration mode, or an alert communicated to the operator via the user indication unit 110. Simple transistor logic may be used with a small ‘ignore time’ to account for possible threshold switching fluctuation.

In alternative embodiments, the coupling component may be configured with parts integrated to minimise size and number of parts. The patient airway interface 202 can be integrated with some or all parts of the coupling component. For example, where the patient airway interface 202 may be in the form of an endotracheal tube, comprises a standard tube and balloon cuff for intubating the trachea at its distal end, and the switch 208 and associated hardware as described above, may be at its proximal end, such that the patient airway interface 202 may be directly linked with the pressure generating unit without the aid of a third component. In this case, the pressure sensitive switch 208 is configured so as to determine the pressure state relative to the threshold pressure inside a lumen of the tube. The valve 220 and exhale port 218 may also be provided in a manner integrated with the patent airway interface 202, allowing exhaled gasses to bypass the pressure generating unit.

So as to allow optional function with existing standard ventilators the coupling component with inbuilt PAI may also be equipped with a ‘bypass’ mechanism, such as a sliding valve or cover able to occlude the exhaust port 218 and incapacitate the directional valve 220 thus forcing expired gases to flow back to the ventilator rather than out into the atmosphere.

In further alternative embodiments, the coupling component may be used to provide positive pressure during the expiration phase of respiration in order to provide PEEP (positive end expiratory pressure). The coupling component may be provided with a user modifiable constriction over the exhaust port such that positive pressure is generated as exhalation is carried out against the constriction.

### Algorithm Components

Algorithms implemented in the computer program code stored in the controller 108 are executable to control the torque and/or speed of the air movement device 100. The algorithms control the air movement device 100 dependent on the binary signal received from the switch 208, and optionally stored information relating to ventilation rate, and optionally any values input by the operator using the first user control 107. The algorithms are also configured to control the user indication unit 100.

As already indicated, a signal received from switch 208 of the coupling component indicates either ‘above threshold pressure’ or ‘below threshold pressure’, manifesting as one of two different binary signals such as a ‘1’ or ‘zero’ or an ‘ON’ or ‘OFF’ such as provided by an ‘open’ or ‘closed’ circuit.

The controller 108 is configured to determine values for variables including the motor throttle value, which also enables electronic braking, and values for control of the user indication unit 110. “Motor throttle” is referred to herein in relation to how the air movement device 100 is controlled, since the air movement device 100 is often in practice a motor. For example, the torque or speed of the motor may be controlled by controlling the motor throttle. The controller 108 may also be equipped with a timer functionality to record the time between any input variable change such as the time between successive switch status changes.

When ventilation is required, a new coupling component (or patient airway interface with inbuilt coupling component) is affixed to the pressure generating unit and all input variables are first set by a user such as: 1.) The ventilation peak pressure (that is, the threshold pressure on the pressure sensitive switch 208) using the second user control; 2.) The desired ventilation rate using the first user control 107; 3.) The desired inspiratory time or inspiratory/expiratory ratio using the first user control 107. After the coupling component is coupled to the pressure generating unit, it is attached to a patient via a suitable patient airway interface 210. Alternatively, the pressure generating unit is directly coupled to a patient airway interface 202 with an inbuilt coupling component. The pressure generating unit may then be secured to the patient, stretcher, bed or other convenient location such that excessive movement is limited. In variant embodiments, the ventilation rate may be preset, for example at 10 breadths per minute. The ratio can also be preset, for example at 1:1. For example, where the ventilation is 10 breadths per minute the inspiratory time is 5 s and the expiratory time is 5 s.

Control algorithms may be pre-programmed within the controller 108 and describe multiple ventilator ‘modes’ that may continuously operate during use. The various modes and their function are described below.

In general, the algorithms begin by calibrating the air movement device in a calibration phase to control parameters to the specific ventilator conditions. The desired peak ventilation pressure has been specified on the second user control 214; however the ventilator must first determine the torque value of the air movement device in order to achieve the desired peak pressure. The calibration phase allows adaptation to many airway and ventilator conditions such as air leaks or varied patient lung physiology.

Once a calibration phase has completed, the ventilator may switch into a normal ventilation mode facilitating normal ventilation whilst continuously self-monitoring to detect any dangerous situations that might arise for the patient. If a potentially dangerous situation is detected the ventilator may attempt to re-adapt to the new conditions by once more going through a calibration protocol. However an attending clinician or other operator may also be alerted if a solution cannot be found by an audible or visual alarm.

### Operation

First, the ventilator is set up for use with the patient. If they are not already, the coupling component and the pressure generating unit are attached. Thus, the first and second mechanical attachment parts 118, 204 are attached together and the first and second electrical interfaces 102, 206 are connected together by the operator. The operator also then uses the dial 214 to set the threshold pressure for the switch 208. The operator may also operate the first user control 107 to set one or more of the variables relating to ventilation. For example, the operator may set a desired breathing rate, and/or an inspiration/expiration ratio.

Alternatively, the patient attachment 222 is then attached to the patient airway interface 200, such that air can be forced into the patient's lungs.

The ventilator may operate in three modes, namely, a calibration mode during set up, a ventilation mode for normal use, and a safety mode.

### Calibration Mode

Characteristics of different patients vary greatly and determining the peak torque at which the air movement device 100 should operate for the particular patient enables the ventilator to take the different characteristics into consideration. For example, different patient's lungs present different lung compliances, different resistances, and have different tidal volume. In addition, possible air leakage between the patient and ventilator necessitates a custom calibration to a patient that varies with patient condition.

In the calibration mode, after the ventilator is set up and the patient attachment 222 is attached to the patent airway interface 202, the controller 108 is configured to determine a peak torque for the air movement device for the particular patient, enabling adaptation of operation of the ventilator to the particular patient. The result is that pressure generated in the patient's lungs does not result in harm, but is sufficient to ventilate the patient. The peak output torque is a proportion of the maximum torque of which the air movement device is capable.

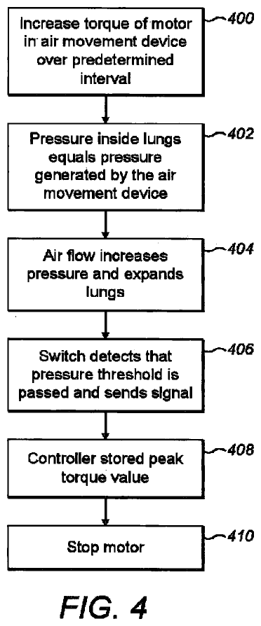

Referring to FIG. 4, regardless of user input settings of the pressure generating unit, in the calibration mode the controller 108 controls at step 400 the electronic speed controller to control the torque of the motor in the air movement device 100, such that a throttle of the air movement device 100 increases according to a programmed function from zero towards a maximum over a prescribed time interval. This results in a steadily controlled increase in pressure in the second conduit 210 in the coupling component and thus also in patient's lungs 202. As the throttle is gradually increased, air flows into the lungs 202 until the pressure inside the lungs equals the pressure generated by the air movement device 100, as indicated at step 402.

The airflow is then zero and the static pressure remains at the pressure generated by the motor. As the throttle increases still further, air flow continues gradually expanding the lungs with the pressure also increasing (step 404). The throttle increase time interval may be predefined in the algorithms and is chosen to be a value that ensures that the pressure as measured within the coupling component is a largely accurate representation of the pressure within the patient's lungs 202 should airflow become static with the motor remaining at a constant RPM. For example, if the time interval is too short, the pressure increases too rapidly within the coupling component without enough time to allow air flow into the patient's lungs thus instantaneous coupling component pressure will be greater than actual intrathoracic pressure. If the throttle increase is too slow, the pressure in the coupling component will be a good representation of intrathoracic pressure however the calibration process will take an unnecessarily long time.

In general, the motor throttle is increased slowly enough to ensure pressure is always effectively equalised between the pressure in the coupling component and the lungs, and that flow is slow enough that there is only a small difference between possible static pressure and current pressure at any given time. In variant embodiments, this time interval may be adjusted by the operator using the first user control 107, but this is not essential. Such adjustment of the time interval is useful in situations where extra time is taken to equalise pressure in the coupling component and in the patient lungs 202; for example this may be appropriate when ventilation occurs through a small calibre conduit such as in neonatal or paediatric ventilation, or ventilation through an emergency tracheostomy such as through a wide bore cannula. In addition, certain lung pathologies may necessitate variable time intervals as discussed below.

As throttle increases, pressure within the second conduit 210 increases resulting in air flow into the patient's airways and thus lungs 202 via the patient airway interface 200. As above, the throttle is increased such that instantaneous gas flow to the patient lungs 202 is low or minimised as sufficient time is given for pressure to equalise. The torque continues to be increased until the pressure in the second conduit 210 just exceeds the threshold pressure set by the operator. This causes the switch 208 to change at step 406 and thus a signal indicating that the threshold pressure has been exceeded to be sent to the controller 108. This results in the controller 108 determining that the threshold pressure has been exceeded. The motor throttle value at this moment is saved in memory of the controller 108 for further use. At this instant, the patient's lungs will contain a maximum volume of air defined by the selected peak pressure as set at the second user control 214 for the switch 208 and the compliance of the lung tissue.

Once the threshold pressure has been reached and the corresponding throttle value for the motor saved, the pressure sensitive switch 208 remains in its ‘tripped’ position in that the determined pressure exceeds its threshold value. This is a dangerous situation as whilst in this ‘tripped’ state the actual pressure value applied to the patient is not known and may exceed what is safe. For example, if there were to be a breathing circuit air leak during the calibration mode throttle rise that was resolved around the time the system reached the threshold pressure, the actual coupling component pressure may rise significantly with no method for detection, thus exposing the patient to a barotrauma risk.

In order to overcome this danger, as soon as the pressure sensitive switch 208 is ‘tripped’ by exceeding threshold pressure, the controller 108 immediately instructs the air movement device 100 to drop throttle to zero thus instantaneously dropping the pressure to near zero at step 410 and allowing exhalation to begin. In an alternative embodiment, the controller 108 begins to gradually decrease the throttle such that the pressure in the coupling component immediately begins to drop. The rate of throttle decrease is programmed such that the defining factor for pressure within the coupling component is the air flow through the air movement device 100.

As soon as the pressure in the second conduit 210 drops below the threshold pressure, the switch 208 trips and the controller 108 receives a signal indicating that the switch 208 has changed change from one state to the other, indicating pressure is now just below the threshold value.

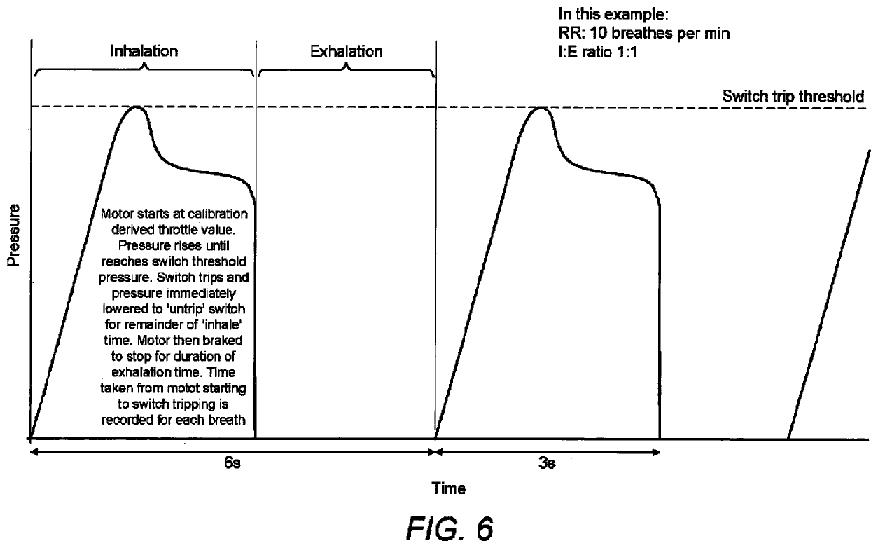

When the switch 208 changes state from above the threshold pressure to below, the pressure in the coupling component is at a value just less than the threshold pressure. The controller 108 then holds the throttle at this new lower throttle value for a defined time period referred to as the ‘inspiratory time’, or the remaining time available for inspiration as defined by the breath rate and the inspiratory/expiratory ratio, as indicated in FIG. 6. Alternatively, the controller 108 may reduce the throttle value to zero, thereby quickly reducing pressure and allowing deflation.

A time limit may be imposed on the above pressure drop operation in order to improve safety and identify hardware faults. If, for example, a significant air leak is fixed, e.g. by making an improved seal to the patient's airway, just after the pressure sensitive switch 208 is ‘tripped’ at the end of calibration mode, the pressure may increase significantly above, risking barotrauma. To avoid this, a time limit may be imposed on the throttle decrease operation. The time limit is defined as the usual time taken for the airflow to decrease as the motor throttle decreases during normal operation. In a case where the throttle is too high risking an overpressure situation, despite throttle decrease in the above way the switch 208 may not reset to its ‘below threshold state’ within the specified time constraints. In this situation, the controller 108 may determine to perform an emergency motor shut off and instruct the air movement device 100 accordingly, thereby preventing the development of an overpressure situation, and also possibly re-start calibration mode in order to re-adapt to the new airway condition. Whilst it is an unlikely situation that a large leak is fixed at the instant the throttle decrease occurs, an alternative explanation for this behaviour is a faulty pressure switch 208 within the coupling component that is incapable of reverting back to its ‘lower than threshold’ pressure state. In the absence of any gas leak, a user may use this safety event as an indicator of a hardware fault and switch over to use a new functioning coupling component.

In a modification, following the pressure sensitive switch 208 trip, the throttle drop phase may begin with an immediate fixed percentage throttle drop calibrated to most likely re-set the pressure sensitive switch 208. If, after a fixed percentage drop and/or a gradual programmed descent, once the pre-configured time has elapsed, if the switch 208 has failed to re-set, either an error must have occurred or new airway conditions are present, resulting in returning to the start of calibration mode to reset the specific threshold throttle value.

Once the threshold throttle value has been reached, the pressure drop carried out within the specified time constraints and the ‘inspiratory time’ throttle hold started, the status of the pressure sensitive switch 208 remains continuously monitored, and should remain in its ‘below threshold pressure’ state.

For the duration of inspiratory time, the pressure sensitive switch 208 should remain in a state at which the pressure is less than the threshold pressure value. If, for example, the calibration mode begins in a situation where there is a significant air leak in the airway system, such as when using a non sealed patient airway interface or in significant facial/airway trauma, the threshold throttle value may be high enough such that, if the flow rate were to be decreased such as if the leak were to be fixed, pressure in the coupling component would exceed the desired threshold value set by the user. If, during the inspiratory time, the switch were to be tripped again, this would imply that air flow conditions have now changed and the current motor throttle value is too high for the current airway scenario. More specifically, this would imply that the pressure in the system has suddenly increased above the desired threshold value despite no change to motor throttle value. This situation risks over-pressurising the patient's lungs as once the pressure sensitive switch 208 is tripped to above the threshold pressure value, there would be no indication as to the extent of possible pressure rise above the threshold value. To prevent this situation, if the pressure sensitive switch 208 is tripped again during the ‘inspiratory time’ the ‘breath’ is immediately aborted such that the throttle value is immediately set to zero and the controller 108 re-starts calibration mode so that the ventilator may re-adapt to the new airway conditions. In certain embodiments, the motor may be electronically braked such that the air flow drops to zero as rapidly as possible.

The calibration mode is deemed to be successfully complete once one full ‘inspiratory time’ or the ‘inspiratory time phase’ has elapsed in the inspiratory/expiratory time ratio, the specific motor throttle value to achieve the threshold pressure has been found and saved, and no safety events have been triggered resulting in re-starting calibration mode or an emergency stop of the motor 100.

In further embodiments, one or more safety stops may be implemented in the calibration mode throttle rise phase as a security measure. During conventional ventilation, it is very uncommon for an operator to desire greater than a maximum pressure of, for example, 27cmH₂O to be delivered to a patient's lungs as the risk of barotrauma and other complications significantly increases. With the air movement device 100, such as a blower motor, it is understood that during unimpeded benchtop study at sea level, any given throttle value is able to generate a maximum possible static flow pressure value in ideal circumstances. In certain ventilation conditions, for example, where airway compliance is extremely low, an operator may choose to ventilate the patient at pressures exceeding the maximum example value of 27cmH₂O. Additionally, during ventilation in the presence of a significant circuit gas leak, it may be necessary for the motor to run at a higher than normal throttle value to meet the desired ventilation pressure. In some cases, this increased throttle value, may, in a static flow situation, exceed the 27cmH₂O safety limit. This is because during an air leak scenario, air flow is never static thus maximum static pressure is never reached. During the initial throttle rise in the calibration mode, if the motor throttle value reaches a pre-programmed value corresponding to a potential maximum static pressure above a safety limit, without triggering the pressure sensitive switch 208, the controller 108 may be configured to cause the user indication unit 110 to alert the user, for example with an alarm or alternative indicator, indicating that user input is required at the first user control 107 to allow any further throttle rise past the safety stop. This safety stop ensures a user does not inadvertently ventilate the patient at a higher than desired pressure, whilst allowing ventilation to take place at higher pressures if circumstances indicate that this is needed. In addition, the safety stop may also identify a coupling component hardware fault. If, for example a user sets the desired ventilation pressure to a value lower than the safety stop and during the calibration mode throttle rise the safety stop is reached, this indicates either a significant air leak must be present which will likely be apparent to a user or there is a fault with the coupling component such as a faulty pressure sensitive switch or a faulty signal transmitting connection. In this situation, the controller 108 is configured to determine this and to alert the user to investigate the possibility of such adverse conditions via the user indication unit 110.

In embodiments, the safety stop is implemented by setting a further threshold above the pressure threshold previously described at the switch 208. The further threshold may be configured within the switch 208 where the switch 208 is configured to output state information indicative of the pressure being above or below a second threshold, that is the further threshold. Alternatively, the further threshold may be implemented with a binary further switch, like the switch 208. The further switch may be co-located with the switch 208, or located elsewhere in the first or second conduits or the patient airway interface, or any region of the ventilator in fluid communication such that pressure is representative of pressure in the lungs. The further switch may be preconfigured to indicate when the pressure exceeds the further threshold pressure and reduces to below the threshold pressure. In operation, the controller 108 receives state information indicative of the further threshold being passed, and takes at least one action based on that information. For example, the controller 108 may cause the operator to be alerted using the user indication unit 110 or to cause the air movement device 100 to allow deflation.

At the end of a successful calibration mode cycle, the lungs will contain a maximum volume of gas as defined by their compliance, the desired ventilation pressure and the inspiratory time. In order to progress to continue ventilation, the motor stops, thus dropping pressure and airflow to equalise with atmospheric pressure for the duration of the expiratory time in order to allow air to passively exit the lungs and vent to the atmosphere via the exhaust port 218.

If calibration mode fails, the controller 108 may initiate a return to the start of calibration mode to re-attempt a calibration attempt. In an example embodiment, after a pre-programmed number of successive unsuccessful calibration attempts, the controller 108 is configured to cause a major alarm to sound to alert the user to attempt to repair any faults or attend to any medical conditions causing the failure.

### Ventilation Mode

This mode is automatically entered once the ventilator has successfully calibrated and controls normal ventilation in stable and unchanging conditions. In the event that the controller 108 detects unsafe change in ventilation parameters, conditions of the ventilation mode become invalid and the ventilator immediately re-enters calibration mode or performs an emergency power off in order to re-adapt to the new situation or prevent any ventilator induced harm occurring. An attending clinician may also be alerted.

Operation of the ventilator is now described when the ventilator is connected to an apnoeic patient requiring full ventilation.

Once sufficient expiratory time has elapsed following end of the calibration mode, the ventilator may now enter ventilation mode and continues normal ventilation until the unit is turned off or any ‘safety’ conditions are triggered requiring an immediate return to calibration mode or total ventilator emergency shutdown. ‘Normal ventilation’ comprises periodic breaths provided to the patient at a rate defined by the operator and at a maximum pressure controlled by the threshold pressure. As mentioned above, the operator may either select a specific ‘inspiratory time’ or they may select an inspiratory/expiratory ratio allowing the actual time spent in each phase to be defined by the selected breath per minute value. During the ventilation mode, the derived specific throttle value obtained during calibration mode and stored at the controller 108 is now used to begin every breath from the outset. The motor of the air movement device 100 is started at the specific throttle value derived in calibration mode, as indicated at step 500, and rapidly begins air flow into the lungs. The pressure in the coupling component gradually increases as the flow rate into the lungs begins to decrease due to the increasing resistance to further distention as volume increases. As the motor continues to run at the known throttle value, some time interval later, henceforth referred to as the dependant variable ‘t’, the lungs are filled to a maximum volume possible at the current throttle value and the airflow rate will correspondingly drop to zero. At this point coupling component pressure will arrive at the threshold pressure thus switching the pressure sensitive switch signal to the ‘above threshold pressure’ state, as indicated at step 502. At this point, the motor throttle is immediately reduced as previously described, such that the pressure switch is reset and the breath continues for the remaining ‘inspiratory time’ or allocated inspiration time available within the inspiratory/expiratory ratio, as indicated at 504. As in calibration mode, a time constraint is imposed on the throttle decrease phase to prevent inadvertent overpressure situations and detect coupling component hardware failure.

The controller 108 immediately reduces the throttle after the switch 208 changes configuration to its ‘above threshold pressure’ state regardless of whether or not the motor has achieved the intended throttle value. If, for example, a significant leak was present during calibration mode such that the saved specific throttle value is very high, if the leak were to be resolved during ventilation mode there is a risk of overpressure due to the new lower airflow conditions thus requiring a lower motor throttle to trip the pressure sensitive switch 208 at its current setting. In this scenario, the switch 208 would trip at a significantly lower motor throttle value then that derived from calibration mode. Once tripped, the throttle would be immediately reduced. However despite the reduction the pressure would not drop lower than the ‘lower than threshold pressure’ switch state within the maximum time thus forcing a return to calibration mode as the previously derived specific throttle value would now be too large for current airway conditions.

Alternatively, a maximum percentage throttle drop may be used rather than a maximum throttle drop time or combinations thereof. Once ‘inspiratory time’ or allocated inspiratory time as per the inspiratory/expiratory ratio has elapsed, the throttle is reduced to zero to allow exhalation to occur. The motor remains ‘off’ for the allocated expiratory time allowing full exhalation to occur prior to restarting the next breath.

At the end of a ventilation mode inspiratory phase, the throttle is set to zero at step 506 (or a low value) and thus the intrathoracic pressure will be greater than the pressure in the coupling component and pressure generating unit pressure. Thus exhaust gas will passively vent to the atmosphere via the exhaust port 218.

Once the expiratory time has elapsed at step 508 and a maximum amount of gas has vented from the lungs the next ‘breath’ of ventilation mode begins thus re-starting the cycle.

As with the calibration mode, the ventilation mode may sometimes proceed at motor throttle values such that the maximum static pressure exceeds the desired threshold value. This occurs in situations where there is an airway leak such that airflow is never zero; thus maximum static pressure for a given throttle value is never reached. As described above, once the pressure sensitive switch 208 is tripped, the throttle is immediately reduced such that the pressure drops until the pressure sensitive switch 208 is re-set to its ‘below threshold pressure’ state. The breath continues for the duration of the inspiratory time or allocated inspiratory time with the switch 208 in this ‘un-tripped’ position. In other words, the pressure in the coupling component is at near maximum, marginally lower than the threshold pressure for the whole of the inspiratory time. If, during the inspiratory time, airway conditions change such that overall air flow rate decreases, the pressure in the coupling component may once again rise above the threshold pressure hence risking an overpressure situation. In this scenario, if the pressure sensitive switch 208 is tripped for a second time during the ‘inspiratory time’ the breath is immediately aborted, controller 108 is configured to cause the user to be alerted via the user indication unit, for example with audiovisual indicators, and the system returns to calibration mode in order to re-adapt to the new ventilation conditions.

If airway conditions change such that a new gas leak develops during normal ventilation mode, the current calibration mode derived specific throttle value may no longer be capable of generating sufficient gas flow and pressure to achieve the threshold value set on the pressure sensitive switch 208. In this case, despite running the motor at the calibrated specific throttle value for the maximum inspiratory time possible the pressure sensitive switch 208 would never change to its ‘above threshold pressure’ configuration. To ensure consistent positive pressure ventilation at the desired value, new leaks may be detected. A gas ‘leak’ may range from extremely small circuit deficits resulting in gas leaks that are easily compensated for by calibration mode, to complete patient disconnects such as the ventilator becoming detached form the patient airway interface 200 during ventilation i.e. a completely open circuit. In addition, the operator may change the desired ventilation pressure by adjusting the dial 214 on the pressure sensitive switch to a higher or lower figure. If the desired pressure is increased during ventilation mode, as above described, the previous specific throttle value may not be able to provide sufficient gas flow to trip the switch 208 at this new setting. If the desired pressure is reduced during ventilation mode, the specific throttle setting will be too high.

Once a ventilation mode breath begins, as described above, the motor throttle is set to the specific throttle value derived during the calibration mode. Pressure begins to rise in the coupling component and the pressure sensitive switch 208 should switch threshold states as the pressure exceeds the threshold value at some point during the inspiratory time. If a new leak develops in the system, the throttle value may not be able to generate enough gas flow and pressure to trip the pressure sensitive switch 208 at any point during the inspiratory time. Thus the whole ‘breath’ will finish without ever achieving the threshold pressure. The controller 108 may then determine airway conditions have now changed and the ventilator will again enter calibration mode to adapt to the new airway conditions. In this example situation, a higher specific throttle value will most likely be needed. The user indication unit 110 may indicate to the user, for example by sounding an alarm, to indicate either an air leak is occurring or, alternatively, the ventilator air intake may be obstructed. If, during the subsequent calibration mode the pressure sensitive switch 208 is unable to be tripped despite multiple attempts at recalibration, this indicates either coupling component hardware failure or significant air leak, the latter being easy for a trained operator to detect, the former requiring a change to a new coupling component.

In operation, the sole feedback to the controller 108 relating to the patient according to embodiments, is the pressure dependant binary output from the switch 208. For example, the signal received might be a 1′ for the duration of time the pressure in the coupling component exceeds the threshold value and a ‘0’ for the duration of time the detected pressure is less than the threshold value. The signal is entirely independent of the numerical value of the pressure at the pressure sensitive switch. The second user control 214 only functions to change the threshold ‘tripping’ pressure. Consequentially, the controller 108 of the pressure generating unit must be programmed with specific algorithm components allowing full safe ventilation to occur using binary signals from the connected pressure sensitive switch.

After use of the ventilator has ended, the coupling component is detached from the pressure generating unit, that is, the first and second mechanical attachment parts 118, 204 are detached and the first and second electrical interfaces 102, 206 are uncoupled. The coupling component is then disposed of and replaced with a new coupling component.

### Safety

1. A set of safety algorithms are features of both the calibration and ventilation modes and constantly run whilst the ventilator is in use. They comprise ‘minor’ and ‘major’ safety alerts and respond appropriately to resolve any potentially dangerous situation through re-calibration, or by stopping the ventilator to prevent harm. Minor alerts may very quickly become major if action is not taken. Safety algorithms that operate in ventilation mode are able to invalidate ventilation mode putting the ventilator into calibration mode when activated, or restarting the calibration mode if activated during the calibration mode process. In critical malfunction situations, they may also completely halt motor function, thus preventing any further gas transfer to the patient. Safety algorithms also assist detection of any hardware component malfunction. Examples of monitored safety parameters are described in detail below and may include but are not limited to:
a. gas leakage though an air leak in the patient circuit
b. gas intake occlusion
c. lung compliance/resistance change manifesting as a change in tidal volume
d. component malfunction
e. component disconnection
f. overpressure protection
g. pressure generating unit malfunction

An overpressure situation is defined as any time where the pressure in the patient's lungs or coupling component exceeds the desired peak ventilation pressure specified by the operator when setting the threshold with the second user control 214. In general, calibration always takes place when the ventilator is first connected to a patient. If the calibration cycle is successful, the ventilator enters ventilation mode.

### Tidal Volume Measurement

A well-recognised disadvantage of IPPV is the lack of ability to factor tidal volume measurement into ventilation. It is crucial to ensure sufficient respiratory gasses enter the lungs during all breaths both to facilitate blood oxygenation and to remove carbon dioxide gas. In normal healthy lungs, the relationship between pressure and volume (expansion) is such that at usual IPPV ventilation pressures (15-20cmH₂O), a sufficient amount of respiratory gasses enters the lungs. In pathological lung states such as adult respiratory distress syndrome (ARDS), pneumo/haemothorax or pulmonary oedema, a ‘compliance curve’ is perturbed such that despite reaching usual positive pressure ventilation pressures (e.g. 15-20cmH₂O), a much smaller volume of gas is moved into the lungs resulting in decreased gas exchange. The lungs behave as if they are significantly ‘stiffer’ as the overall lung compliance decreases i.e. a larger pressure change for a given volume change is required. Conversely, if an intervention is carried out to reverse pathology such as a chest drain insertion during a haemothorax, a clinician will want to see a corresponding increase in compliance indicating a successful intervention. As tidal volume increases or decreases, there is a need to adjust desired respiratory pressures to ensure gas exchange remains optimal for the current clinical situation. For example, if tidal volumes and lung air entry as auscultated via a stethoscope gradually decrease despite a constant desired pressure setting, a clinician might assume some form of respiratory pathology is present. In order to ensure satisfactory gas exchange continues, the clinician may increase the ventilation pressure threshold using the dial 214 until tidal volume once again increases.

Existing electronic ventilator technologies utilize multiple digital pressure sensors arranged to detect precise flow rates and volumes of gasses entering and exiting the patients lungs. This precision comes at the cost of expensive sensors, complex software and calibration procedures adding to the high price and poor resilience of existing ventilators. Certain pneumatic ventilators may also show tidal volume using either complex pneumatic mechanisms or integrated digital sensors. Like fully digital systems, this increases the cost and complexity whilst decreasing reliability and robustness. Whilst it is advantageous to calculate exact lung tidal volumes, in the acute critical care situation, tracking a ‘change’ corresponding to clinical findings rather than absolute values may be adequate.

In embodiments, changes in tidal volume may also be indicated without the use of any digital sensors or complex electronic components or software. During ventilation mode, as described above, the air movement device 100 is operated at the calibration mode derived specific throttle setting. Assuming constant airway conditions, whilst the air movement device 100 is immediately started at a specific throttle, some time period ‘t’, will elapse before the coupling component pressure climbs to the threshold value to trip the pressure sensitive switch 208 as some time interval is required to allow the lungs to fill with gas. If leak conditions remain constant, the time ‘t’ will depend only on the rate at which air escapes the coupling component into the lungs assuming the air movement device 100 operates in a consistent manner at a throttle value derived from calibration mode. As the lung volume and pressure increase the overall coupling component flow rate will decrease resulting in a corresponding pressure increase towards the threshold pressure. The controller 108 may operate to record the time taken from the moment the air movement device 100 starts at the beginning of a breath to the point of change of threshold state of the pressure sensitive switch 208. This time period is recorded as the value ‘t’ and may be saved in the memory unit of the controller 108 of the pressure generating unit.

If some pathological process occurs inducing either a decreased lung compliance or decreased lung volume such as a pneumothorax or ARDS, the lungs appear to become ‘stiffer’ with respect to the ventilator such that for a given pressure rise, less air flows into the lungs. This means that given a constant throttle value, the time taken to achieve the threshold pressure will decrease in some proportion to the decrease in compliance. If ‘t’ is sequentially recorded for every breath during ventilation mode, the trend of the value of ‘t’ may be interpreted as the trend in lung tidal volume thus compliance. For example, if, during ventilation mode ‘t’ begins to decrease it can be assumed that some pathological process is decreasing the compliance of the lungs. Accordingly, preferably the controller 108 is configured to determine intervals between breaths as indicated by when the pressure rises above threshold. Preferably the controller 108 is configured to determine a trend indicating decrease in compliance and to indicate such to the user using the user indication unit 110. The controller 108 may also determine to re-enter calibration mode in this case. To counter this, the clinician can increase the desired threshold pressure on the coupling component to further increase the tidal volume and achieve satisfactory air entry according to their clinical findings.

In this embodiment, whilst it is not possible to determine the ‘normal’ tidal volume for a given patient, the tidal volume variability can be interpreted with reference to clinical findings to manage any pathology that arises. For example, if the ventilator unit were to be affixed to a patient already suffering from significantly decreased lung compliance, the success of any intervention can be indicated by observing an increase in ‘t’ corresponding to an increase in the time taken to ‘fill’ the lungs due to the intervention mediated newly increased compliance.

It is understood that the value of ‘t’ can be displayed on any number of differing graphical user interface solutions such as an LED light strip or digital number read out. For example, when the ventilator is first initiated, the ‘t’ value is derived and may be displayed as a ‘middle’ value. If compliance increases or decreases during ventilation the T value indicator may correspondingly increase or decrease. An option may be provided to ‘re-zero’ the ‘t’ value should the clinician wish if, for example, the ventilator is initiated on a patient with clear respiratory pathology that is rectified by the clinician enabling the ‘t’ indicator to be brought back into the ‘middle’ to best pick up any further changes.

In certain embodiments, the numerical value of ‘t’ may be indicated in conjunction with fixed pressure generating unit motor throttle values and known patient airway interface 212 in order to provide a ‘inspiratory compliance metric’ to scientifically compare the compliance of a patient's airways and provide an indicator as to how far from ‘normal’ values a patient may be.

It is understood by those skilled in the art that whilst accurate tidal volumes cannot be derived, monitoring change may be acceptable to an attending clinician considering the significantly increased simplicity of the measuring apparatus.

A change in the ‘t’ value may also be used to indicate other pathology or ventilator status metrics. For example, an increase in airway resistance can occur in conditions such as anaphylaxis or severe asthma. An increase in resistance results in an increase in the value of ‘t’ as slower air flow results in a longer time to reach a zero flow condition. It is recognised, however, that inaccuracies and tolerances in conventional ventilators result in the ‘t’ value decreasing with increased resistance as the reduction in airflow manifests in much faster times to reach approximately peak pressure at which the pressure sensitive switch 208 detects this approximate switching pressure and ‘trips’.

### Further Safety Embodiments

During calibration mode or ventilation mode, failure of the pressure sensitive switch 208 may pose a significant risk to patient safety. For example, a failure situation is most likely to occur in one of two ways: failure to trip at the correct pressure and failure to ‘re-set’ despite an appropriate reduction in pressure.

The former failure situation poses a risk of barotrauma and ventilation at too high a pressure compared to the desired pressure indicated on the dial 214, whereas the latter risks total ventilator malfunction but no barotrauma risk.

In one embodiment, the aforementioned further threshold, in the form of a safety stop(s), during calibration mode may act to prevent damage from this failure situation. Assuming a coupling component with faulty pressure sensitive switch 208 is connected to the pressure generating unit, during the calibration mode the pressure will steadily rise and may travel past the desired set point entered on the dial 214. As a faulty pressure sensitive switch 208 is unable to trip, the calibration mode throttle rise will continue until the throttle percentage reaches the safety stop cut off and the operator is then alerted. As previously described, the safety stop occurs at a high enough pressure at which it would be uncommon to ventilate but not dangerous to a patient. The clinician would examine the ventilator set up and observe that despite no significant air leakage the safety stop alarm is still triggered. The vast majority of the time, the required ventilation pressure set on the coupling component will be lower than the safety stop thus in the absence of a leak the operator may deduce that the pressure sensitive switch 208 failure has occurred and the coupling component must be swapped. If the ventilator is being used where leaks are present the operator may detach the ventilator and place a finger or stopper directly over the air output aperture. The calibration mode is then initiated in a known ‘no leak’ situation and the above checking procedure may be followed. If the same situation occurs in a known ‘no leak’ scenario, it is deduced that the coupling component is at fault and should be replaced.

The latter pressure sensitive switch failure situation may be detected with an addition to the aforementioned safety algorithm. Once the pressure sensitive switch 208 is tripped either during calibration mode or ventilation mode, the throttle is immediately reduced such that the pressure sensitive switch 208 re-sets. If the throttle value has been reduced beyond a cut off threshold without re-setting the switch the motor throttle is dropped to zero. Once throttle is at zero and the motor is stationary, or the throttle is at a small throttle percentage, if the switch 208 remains in the above threshold pressure state this signifies a pressure sensitive switch 208 failure prompting an alarm to alert the operator to swap the faulty coupling component.

In a further embodiment, the coupling component may be provided with a yet further pressure sensitive switch, additionally or alternatively to the further pressure switch, within the coupling component arranged such that it may detect a small negative pressure within the coupling component. This detection allows the unit to operate in a ventilator support modality. If a patient is attempting to spontaneously ventilate with insufficient effort, a ‘patient demand’ mode may be entered where breaths are triggered by the patient. As the patient attempts to inhale through the coupling component, the pressure falls and is detected by the yet further pressure sensitive switch in this embodiment. This triggers a single breath operated by the ventilation mode algorithm. The yet further pressure sensitive switch may be co-located with the pressure switch 208 and/or the further pressure switch, or located elsewhere in the coupling component where the pressure is representative of lung pressure.

In another embodiment, the coupling component may be provided with mounting ports for any prior art ‘end tidal’ carbon dioxide monitoring system and a gas flow measurement system. It is understood that these digitally measured features are present on many current digital ventilators. Whilst the simplicity of the above described design is unable to directly measure these parameters, a facility is provided to affix an existing carbon dioxide monitor and flow measurement apparatus onto the coupling component to provide optional, in depth information to a clinician should it be desired e.g. using the ventilator in an anaesthesia delivery setting.

In another embodiment, it is recognised that during ventilation, gasses with a high percentage composition of oxygen are desirable. The pressure generating unit may be provided with an inlet aperture either at the intake of the air movement device 100 or at the output thereof onto which an oxygen delivery device may be affixed via, for example, a tube. In this embodiment, an oxygen delivery device may comprise an oxygen gas source, a reservoir bag and a valve that allows gas to flow from the reservoir bag into the pressure generating unit. Oxygen gas may run at a constant flow rate from the source. Flow rate may be checked in a look up table and set such that the desired percentage composition of oxygen is delivered to the patient. During gaps between breaths, oxygen gas flow enters the reservoir bag filling it with oxygen as the motor is not moving air thus no gas is being drawn into the ventilator air intake. Once a new breath begins, low pressure at the motor air intake preferentially draws gas from the oxygen reservoir through the motor and to the patient. Once the reservoir bag is empty, atmospheric air continues to flow through the system.

A further safety feature would come into play should the pressure generating unit CPU malfunction and the air movement device 100 may operate to move air in an uncontrolled manner risking patient injury. The pressure generating unit may be provided with an electrical current sensing apparatus in line with the motor power wires, capable of severing power to the air movement device 100 with, for example, an electrical relay or transistor. During ventilation mode, if, for any reason the electrical current to the motor were to surge more than some multiple of the average current since the last calibration cycle, power would be disconnected to the motor to protect the patient.

In a further embodiment, it has previously been discussed that a new coupling component should be used with each new ventilated patient to ensure reliability and safety of ventilation. In an emergency, if coupling component failure occurs and no replacement was available, the pressure generating unit may operate in a ‘salvage’ mode that allows some form of rudimentary ventilation without any detection facilities. When in salvage mode, the pressure generating unit may operate without any coupling component input. As previously described, during benchtop testing the air movement device may be operated at varying throttle levels and at each throttle level a maximum peak static pressure is possible. Anybody skilled in the art will understand a study may be carried out on the pressure generating unit at normal atmospheric pressure and a corresponding throttle value identified at which the maximum static pressure is, for example, 20cmH₂O. During salvage mode, a standard ventilation pressure of, for example, 20cmH₂O is chosen and the corresponding throttle value is pre-programmed into the controller 108. Once the controller 108 determines to initiate salvage mode, the air movement device 100 is caused to operate by the controller 108 to intermittently turn on and off at the pre-programmed ‘salvage’ throttle value at a rate defined by stored respiratory rate, which may have been input or may be adjusted with the first user control 107. In this way, in a worst case scenario situation, a patient may still be ventilated at pressures, approaching but not exceeding the pre-set salvage mode pressure. It is, however, noted that no sensing or safety features would be functional in this modality.

In a still further embodiment, the hardware components may be operated to provide continuous positive airway pressure (CPAP) for a patient. Conventionally, CPAP is used to provide ventilatory support to a patient who remains able to independently breathe but requires ‘non-invasive support’.

To begin CPAP mode, the first user control 107 may be positioned to an indicated setting. In this mode, the operator begins by adjusting the pressure sensitive switch 208 to indicate the desired CPAP pressure setting. Calibration mode then begins and determines the specific throttle value required to trip the switch 208 as previously described with the current airway conditions. Once this specific throttle value is derived, the throttle is dropped until the switch 208 re-sets and this value is held. If airway conditions change such that the switch 208 were to once again trip indicating an unintended rise in pressure, the controller 108 immediately reduces throttle at the aforementioned rate for the maximum prescribed time. If, despite this, the switch 208 remains tripped, the CPAP is aborted and returns to calibration mode to determine the new airway conditions. During normal operation, the controller 108 periodically raises the throttle back to the calibration mode derived specific throttle value (plus overshoot if used). During this rise, the switch 208 should trip again provided airway conditions have remained constant. If, for example, a new leak has occurred, despite a rise to the calibration mode derived specific throttle value (with overshoot) the switch 208 would not re-trip. This eventuality forces an immediate return to calibration mode in order to re-calibrate to the new airway conditions.

In a still further embodiment, lung airway resistance may be inferred using the calibration mode throttle rise. Lung resistance is an important metric as many respiratory pathologies manifest with changes in airway resistance such as asthma or anaphylaxis attacks.

In these situations, compliance may be normal thus tidal volume may stay the same however due to the narrowed airways a longer time increment is required to fill the lungs to their maximum tidal volume. As previously described during calibration mode, once the switch 208 has been tripped for the first time, the throttle is decreased such that the switch 208 is restored to its ‘below threshold pressure’ setting and the air movement device 100 is held at this constant reduced throttle setting for the duration of inspiratory time. If the patients lungs have a high airway resistance, despite the appearance of the switch 208 tripping due to reaching largely static airway conditions at the end of calibration mode, air flow will, in fact, not have finished passing into the lungs. This effect may be seen as a new sudden increase in pressure during the inspiratory time throttle hold causing the pressure sensitive switch 208 to trip again. In addition, increased airway resistance may be predicted by an attending clinician. For example, if ventilation was desired through a small conduit such as an emergency improvised airway or a small calibre endotracheal tube, inherent airway resistance would be very high.

High resistance airway scenarios such as this can be alleviated by slowing the calibration mode throttle increase to better match the specific throttle value to the airway conditions. An adequate throttle increase speed is indicated once a full inspiratory time is able to elapse without the pressure sensitive switch 208 tripping a second time.

The transthoracic pressure differential is the most important factor defining actual lung ventilation pressures. For example, if a patient is ventilated at a pressure sensitive switch setting of 20cmH₂O at considerable altitude, due to the lowered ambient atmospheric pressure the actual transthoracic pressure differential at the point of pressure sensitive switch ‘tripping’ will be significantly more than 20cmH₂O. For this reason, an altitude compensation table may be provided in order to adjust the selected pressure sensitive switch threshold pressure setting to correspond to the desired ventilation pressure whilst taking into account the ambient atmospheric pressure that may be decreased due to altitude. Alternatively, a coupling component may be provided additionally comprising a prior art barometric pressure sensor mechanically connected to the pressure sensitive switch control dial. The barometric sensor may alter the positioning of the dial graduations according to the measured barometric pressure such that the user will always inadvertently select a desired pressure differential that reflects the ambient barometric air pressure.

In another embodiment, the invention may be retrofitted to existing prior art ventilators to provide a backup ventilation system. If, for example, an existing digital ventilator comprising multiple digital or mechanical pressure sensors were to malfunction, the ventilator controller may automatically switch to a backup pressure sensitive switch positioned within the patient circuit and operate the ventilator as described above as it is less likely such a system would malfunction.

In a still further embodiment, whilst the values of the variable ‘t’ may be used to infer changes in the tidal volume and thus compliance (and resistance), drastically variable values of ‘t’ may be used to indicate a patient that is ‘fighting’ the ventilator. This means the patient does not have a sufficiently low level of consciousness to tolerate forced invasive ventilation. The ‘t’ value would vary drastically as the patient would independently take their own breaths at non synchronised time periods. If, for example the patient were to attempt to exhale whilst the ventilator attempted to provide a breath, the pressure would rapidly increase causing the pressure sensitive switch 208 to rapidly trip. If, on the subsequent breath the patient were to be at a different phase of spontaneous ventilation such that the lungs are filled as normal the value of ‘t’ would increase again. In this case a solution may be to switch the patient to a coupling component that is configured with a switch, as mentioned above, to detect a small negative pressure threshold such that the controller 108 is able to detect when a small negative pressure is applied by the patient and provide a supported breath.

In further embodiments, it is recognised that multiple methods of controlling an air movement device electric motor exist in the art. For example, in the control of a brushless direct current motor, systems may employ a ‘closed loop’ or an ‘open loop’ architecture. Many control architectures must inherently calculate the instantaneous RPM of the motor in order to synchronise magnetic coil changes. In some embodiments, the controller 108 of the pressure generating unit may utilise an exact reading of motor RPM to further refine ventilator control.

In an alternative configuration, the parts comprising the coupling component may be directly built into the pressure generating unit such that the ventilator may comprise a single discreet unit. In this case a patient airway interface may directly connect to the pressure generating unit. This component may be designed such that individual components are replaceable such as the exhaust valve or pressure sensitive switch. Conversely, if the disposable unit is to be separate from the pressure generating unit they may be deployed in variable locations. For example, in one embodiment, the coupling component may be affixed directly to a patient airway interface and then directly connected to the pressure generating unit. In another embodiment, the two parts may be remote from one another connected via an airway conduit comprising a gas flow channel and a means to communicate the pressure sensitive switch threshold signal.

As may be appreciated in view of the above disclosure, an aim of embodiments is to enable the coupling component to have parts that are inexpensive and reliable. Accordingly, a simple pressure switch is used, whereas in prior art ventilators much more complex switches are used. Embodiments of the invention may include a plurality of binary sensors. Embodiments may also include simple sensors that are configured to detect changes of pressure across multiple thresholds. Such embodiments are within the scope of the invention.

It will be appreciated by persons skilled in the art that various modifications are possible to the embodiments.

In variant embodiments, the pressure generating unit may include one or more additional measuring devices, for example to measure barometric pressure, coupled to the controller 108. The controller 108 may also control operation of the air movement device 100 dependent on such other measured variables. It is generally known in the art that further variables may be monitored and used in controlling operation of a ventilator, and detailed discussion is outside the scope of this disclosure. Information indicative of such further variables may also be displayed by the user indication unit 110.

The controller 108 may be configured to obtain state information from the switch 208, or any further or yet further switch mentioned above by sending of an interrogating signal to the relevant switch and receiving a response signal. Alternatively, the relevant switch may be configured with functionality enabling sending of such state information without first receiving a signal from the controller 108. Notably, where signalling is initiated by the switch, it is inessential for the signal to be regular. The relevant switch need only indicate when its state is changed. Where state is monitored at the controller 108, the only information required from the relevant switch need be information indicative that a change has occurred.

No limitation is to be construed by titles herein. The applicant hereby discloses in isolation each individual feature or step described herein and any combination of two or more such features, to the extent that such features or steps or combinations of features and/or steps are capable of being carried out based on the present specification as a whole in the light of the common general knowledge of a person skilled in the art, irrespective of whether such features or steps or combinations of features and/or steps solve any problems disclosed herein, and without limitation to the scope of the claims.

1. Apparatus for a ventilator, comprising:
a control unit operatively coupled to an air movement device to control the air movement device, and to receive state information Indicative of pressure being above or below a threshold pressure from a pressure switch, wherein, in use, the pressure switch is located at a pressure representative of air pressure in lungs of a patient, and wherein the state information does not comprise and is independent of an absolute numerical pressure valve for the pressure sit the pressure switch, wherein the control unit is configured to:
a) control the air movement device to cause inflation of the lungs;
b) receive the state information indicative of pressure being above or below the threshold pressure;
c) based at least on the state information indicating that the pressure is above the threshold pressure and on stored information relating to an inspiration period and an expiration period, control the air movement device to enable expiration, and start a timer for an expiration period;
d) determine that the expiration period has ended based at least on the stored information relating to the expiration period, and in response thereto to repeat a) to d).

2. The apparatus of claim 1, comprising a first part and a second part, wherein the first part includes the control unit, and the second part includes the pressure switch, wherein the second part is detachably attachable to the first part.

3. The apparatus of claim 2, further comprising a conveying component configured to convey the state information from the pressure switch to the control unit.

4. The apparatus of claim 3, wherein the state information conveying component comprises a first information conveying portion in the first part and a second information conveying portion in the second part, wherein the first and second information conveying portions are detachably attachable.

5. The apparatus of claim 2, further comprising: conduit unit comprising:
a first conduit part in the first part of the ventilator coupled to the air movement device; and a second conduit part in the second part of the ventilator, wherein the first and second conduit parts are connectable such that in use air is caused to flow from the first conduit part to the second conduit part, and wherein the first and second conduit parts are detachably attachable;
wherein the second part further comprises a patient interface coupled to the second conduit part and configured for attaching to the patient such that air can be caused to flow into the lungs of the patient.

6. The apparatus of claim 1, wherein the pressure switch is located in the patient interface.

7. The apparatus of claim 2, wherein the second part comprises:
a valve in the second conduit part; and
an air outlet coupled to the valve, wherein the valve is configured to permit flow of air from a first side of the valve nearer to the air movement device to a second side of the valve nearer to the patient when air pressure at the first side is greater than at the second side, and to cause air to flow from the second side through the air outlet when air pressure at the second side is greater than air pressure at the first side.

8. The apparatus of claim 1, further comprising a first user control coupled to the pressure switch for setting one or more threshold pressures for the pressure switch.

9. The apparatus of claim 1, further comprising a second user control coupled to the control unit, configured to enable input of the stored information relating to the inspiration period and the expiration period, wherein the stored information is indicative of at least one of: breathing rate, inspiration period, expiration period, and an inspiration-to-expiration ratio.

10. (canceled)

11. The apparatus of claim 1, wherein:
step a) the air movement device is configured to start the inspiration period when starting to cause the inflation; and in step c) in response to the state information indicating that the pressure is above the threshold pressure, the control unit is also configured to determine that the inspiration period is ended, wherein the expiration period is dependent on the inspiration period and the breathing rate information.

12. The apparatus of claim 11, wherein the control unit is further configured to determine duration of inspiration until the pressure threshold is exceeded, store information indicative of the determined duration and to determine a change in compliance of the lungs based on a predetermined change in the durations over different inspiration periods.

13. The apparatus of claim 1, wherein the control unit is configured to:
receive further slate information from the pressure switch or a further pressure switch indicating that the pressure at the pressure switch is below a further threshold pressure;
further to receiving the further state information indicating that the pressure is below the threshold pressure, control the air movement device to cause pressure between the air movement device and the patient to rise by increasing the power supplied to the air movement device;
in response to receiving state information indicating that the pressure exceeds the threshold pressure, setting a maximum power value to use with the air movement device.

14. (canceled)

15. A computer program product comprising computer program code stored on a memory unit, which, when run on a processing unit is configured to cause the processing unit to perform the steps of:
a) controlling the air movement device to cause inflation of the lungs;
b) receiving state information indicative of whether pressure is above or below at least one predetermined threshold pressure at a pressure switch, wherein, in use, the pressure switch is located at a pressure representative of air pressure in lungs of a patient, and wherein the state information is independent of and does not comprise an absolute numerical pressure at the pressure switch;
c) based at least on the state that the pressure is above the threshold pressure and on stored information relating to an inspiration period and an expiration period, controlling the air movement device to finable expiration, and start a timer for an expiration period;
d) determining that the period has ended based at least on the stored information relating to the expiration period, and in response thereto to repeat a) to d).

16. (canceled)

17. The computer program product of claim 15, the threshold pressure value, wherein the steps further comprise receiving the threshold pressure value input by a user at a user control.

18. The computer program product of claim 15, wherein the steps further comprise:
when the pressure is below the threshold pressure, controlling the air movement device to cause pressure between the air movement device and the patient to rise by supplied to the air movement device;
in response to receiving state information indicating that the pressure exceeds the threshold pressure, setting the maximum power output of the air movement device as the maximum power for use with the air movement device with the patient.

19. The computer program product of claim 15, wherein the steps further comprise: storing information relating to an inspiration period and an expiration period, wherein the information is indicative of at least one of: breathing rate, the inspiration period, the expiration period, and an inspiration-to-expiration ratio.

20. A method of operation of a ventilator, comprising, at a control unit:
controlling the air movement device to cause inflation of the lungs;
b) receiving information from a pressure switch indicative of whether pressure is above or below a predetermined threshold pressure, wherein the pressure sensor is located in air at a pressure representative of air pressure in lungs of a patient, and wherein the information does not comprise and is independent of an absolute numerical pressure at the pressure sensor;
c) based at least on the state information indicating that the pressure is above the threshold pressure and on stored information relating to an inspiration period and an expiration period, controlling the air movement device to enable expiration, and start a timer for an expiration period;
d) determining that the expiration period has ended based at least on stored information relating to the expiration period, and in response thereto to repeat a) to d).
